(19) United States
(12) Patent Application Publication
(10) Pub. No.: US 2004/0044341 A1
(43) Pub. Date: Mar. 4, 2004

(54) Electrosurgical device and method of use

(76) Inventors: Csaba Truckai, Saratoga, CA (US); John H. Shadduck, Tiburon, CA (US)

Correspondence Address: Csaba Truckai, 2629 B Terminal Boulevard, Mountain View, VA, 94043, US

(21) Appl. No.: 10/228,857

(22) Filed: Aug. 27, 2002

(51) Int. Cl. A61B 18/18

(52) U.S. Cl. 606/41

(57) ABSTRACT

This invention relates to electrosurgical systems and techniques for applying ohmic heating to tissue, and for shrinking a dimension across a tissue surface. More particularly, the invention provides a tissue-conforming tape or patch member for conforming to and adhering to the surface of a targeted body structure. The tape or patch can be coupled to an electrical source to thereby apply Rf energy to a conductive engagement portion of the conforming member. The conforming member can be used to controllably cause ohmic heating in the engaged tissue to shrink, coagulate, ablate or create lesions in the body structure.

### BACKGROUND OF THE INVENTION

1. Field of the Invention

This invention relates to electrosurgical devices and techniques for applying energy to tissue for hemostasis, coagulation or tissue-shrinkage purposes. More particularly, the system provides an electrosurgical “tape” or “patch” that consists of a thin surface-conforming member that can be applied to the surface of a targeted body structure. The system provides mono-polar or bi-polar electrode means within an engagement surface for active Rf energy delivery from the conforming member that conforms and adheres to an organ's surface (e.g., a liver or lung).

2. Description of the Background Art

Various devices and techniques have been developed for coagulation or sealing broad surface areas of tissues or organs. For example, argon coagulators are known wherein an ionized gas serves as a gas electrode that is sprayed over a targeted site. Such argon coagulation relies on a first polarity electrode at the instrument working end delivers energy across the gas electrode in cooperates with a ground pad serving as a return electrode. In argon coagulation, the depth of ohmic heating in the tissue surface is not controllable since surface desiccation causes localized high impedances. What is needed is an improved means to cause ohmic heating in tissue to a controlled depth with a conductive electrode that conforms to the targeted tissue surface.

### SUMMARY OF THE INVENTION

This invention relates to electrosurgical systems and techniques for applying ohmic heating to tissue. More particularly, the invention provides a tape or patch member for conforming to, and adhering to, the surface of a body structure. The tape or patch member then can coupled to a electrical energy source for applying Rf energy to a conductive engagement portion of the tape or patch, which in turn will controllably cause ohmic heating in the engaged tissue surface to shrink, coagulate, ablate or create lesions therein.

The method of the invention is useful for applying energy to surface areas of an organ or other anatomic structure. At the same time, the tape, patch or pad can provide a sealing film over the treated region. Also, the conforming member can carry a pharmacologically active agent for delivery to the treatment site. Further, the tape or patch can be fabricated, at least in part, of a heat-shrink polymer that can contract a selected dimension of the engaged tissue surface.

The use of the thermal shrinkage aspect of the tape or patch can be useful for both thermally shrinking tissue and mechanically contracting the engaged tissue is altering the elastic and dimensional parameters of a patient's pelvic floor. The use of thermal shrinkage of the tape or patch also can be useful in a lung volume reduction surgery wherein the conforming material is folded or wrapped around a targeted lung segment—and the conforming device of the invention collapses, compresses and seals the substantially surrounded lung segment to thereby reduce to overall lung dimension to allow other non-treated portions of the lung to function better.

The invention advantageously provides a tissue surface conforming member with a bi-polar electrode system that can be used to deliver bi-polar Rf energy to tissue.

The invention provides a system and method for creating a bi-polar electrode that perfectly conforms to irregular tissue surfaces.

The invention provides a method for controllably delivering Rf energy to a selected depth in tissue by (i) controlling the center-to-center distance between spaced apart tissue-surface conforming electrodes, and for (ii) controlling the rate of energy delivery between the spaced apart electrodes.

The invention provides devices and methods for controllably shrinking and collapsing an engaged tissue volume such as a lung.

The invention provides devices and methods for controllably coagulating and sealing an organ surface such a patient's liver.

The invention provides devices and methods for creating controlled depth lesions in tissue, such as in pulmonary vessels to alter conduction pathways.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout the disclosure.

FIG. 1 shows a plan view of an exemplary hand-held instrument with a working end that dispenses a Type “A” conforming electrosurgical device or tape member corresponding to the invention.

FIG. 2 is an enlarged cut-away view of the conforming electrosurgical member of the invention.

FIG. 3 depicts multiple layers of the conforming electrosurgical tape member over a targeted site.

FIG. 4 is a bi-polar embodiment of the conforming electrosurgical tape member of FIG. 2.

FIG. 5 illustrates an alternative Type “B” embodiment of the conforming electrosurgical patch member and its method of use.

FIG. 6 illustrates a bi-polar embodiment of the conforming electrosurgical member of FIG. 5 and its method of use.

### DETAILED DESCRIPTION OF THE INVENTION

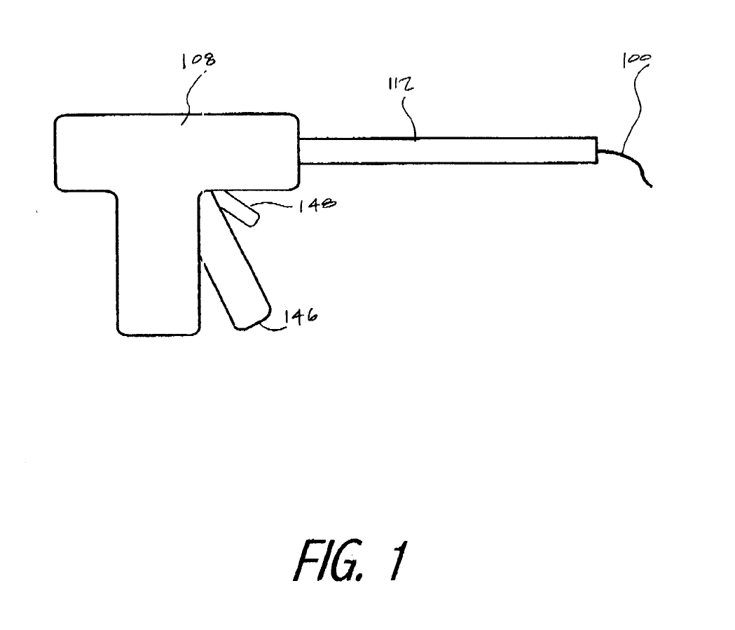

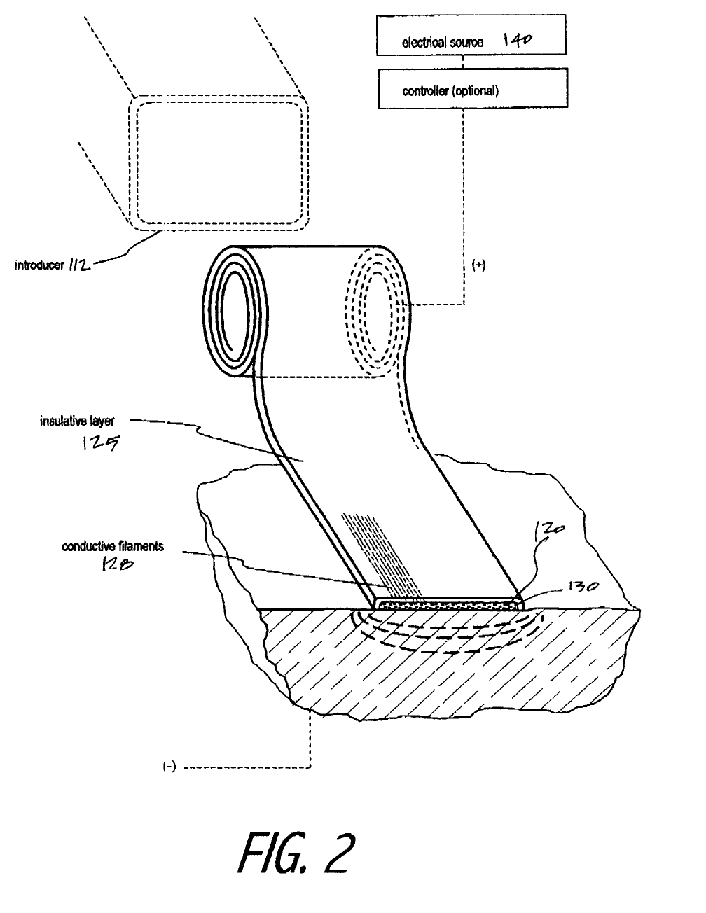

1. Type “A” embodiment of surface-conforming electrosurgical device tape. FIGS. 1 and 2 illustrate a Type “A” electrosurgical device or tape 100 corresponding to the invention for creating ohmic heating in a tissue surface, after adhering to the surface. The handle portion 108 and introducer member 112 in FIG. 1 are an optional component of the system for dispensing tape 100 as shown in FIG. 2, for example in an endoscopic surgery. In an open surgery, the tape or patch can be applied manually—and is cuttable and trimmable with a scissors.

Of particular interest, the tape member 100 is adapted to completely conform to, and adhere to, the surface of a targeted body structure T in FIG. 2, which for example can be the surface of a liver. The tape 100 can be used to controllably cause ohmic heating of a tissue surface to coagulate, ablate, shrink or create lesions in the targeted surface. At the same time, the polymer tape member can have provide a sealing member with a controlled porosity, or no porosity, in the device. A microporous tape member also can carry any desired pharmacological agent for delivery to the tissue. The layers of the device 100 also can be substantially of a biodegradable polymer.

As can be seen in FIG. 2, the tape 100 has a first surface portion 120 that comprise a flexible conductive material that defines an engagement surface 122 for engaging tissue. The tape 100 has second surface 125 that comprises a flexible insulative material. In one embodiment as shown in FIG. 2, the flexible conductive material comprises conductive filaments 128 in a conductively doped polymer 130. The tape 100, and more specifically, the conductive filaments 128 are coupled to electrical source 140. Conductively doped plastics are known in the art, and for example can be doped with carbon particles.

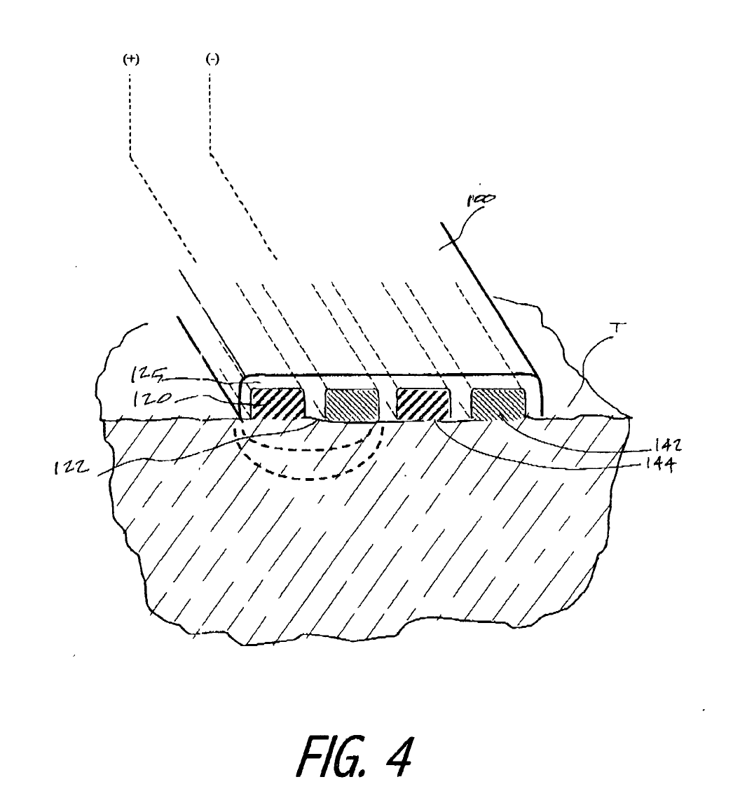

In another embodiment depicted in FIG. 4, the tape 100 has a first surface portion 120 that carries a plurality of spaced-apart flexible conductive portions indicated at 142 and 144. Each such portion can be a conductive polymer, and each may have its own conductive filaments therein that are coupleable to an electrical source. These conductive portions 142 and 144 can operate as spaced-apart bi-polar electrodes to control depth of ohmic heating—which is largely a function of center-to-center spacing of the electrodes. A multiplexer can be provided to apply Rf energy between selected various spaced-apart electrode groups to further control depth of ohmic heating.

In a preferred embodiment, the tape 100 has a second insulative surface 125 of a transparent polymer. The tape 100, and each of its layers, can be of any suitable flexible polymer, such as a polysiloxane, polyurethane, PFTE, polyacrylate, polyamide, polyester, polyolefin, nylon or any co-polymers thereof. In one preferred embodiment, the tape 100 is stretchable. In another preferred embodiment, the tape 100 is elastic. In a typical embodiment, the tape 100 is foldable and deformable to adhere to irregular tissue surfaces. In another preferred embodiment, either or both the first and second surfaces of the tape member 100 carry a thermochromic composition to provide a visual indicator of temperature of the tape and the engaged tissue.

The tape 100 can carry any suitable adhesive composition on its engagement surface 122. The adhesive can be a cyanoacrylate glue, or a fibrin-carrying glue.

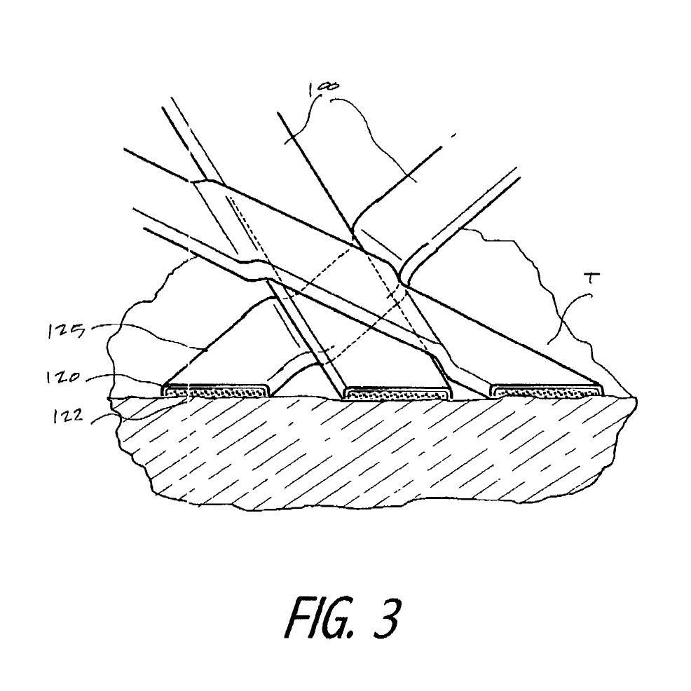

In FIG. 3, it can be seen that many multiple layers of the tape 100 can be applied over each other to engage broad tissue surfaces. This is a particular advantage of providing the insulative layer 125 over the conductive layer 120, particularly in the bi-polar tape version of FIG. 4.

FIG. 1 shows a handle member that serves as a dispenser of tape 100. It can be understood how the lever 146 can operate an internal ratchet to rotate a tape spool of FIG. 2 to dispense tape. The lever 146 can serve as a dual-acting actuator wherein a full squeeze of the lever cuts the tape after Rf energy delivery with a blade or wire element. The trigger 148 can be used to turn on the Rf delivery from the electrical source.

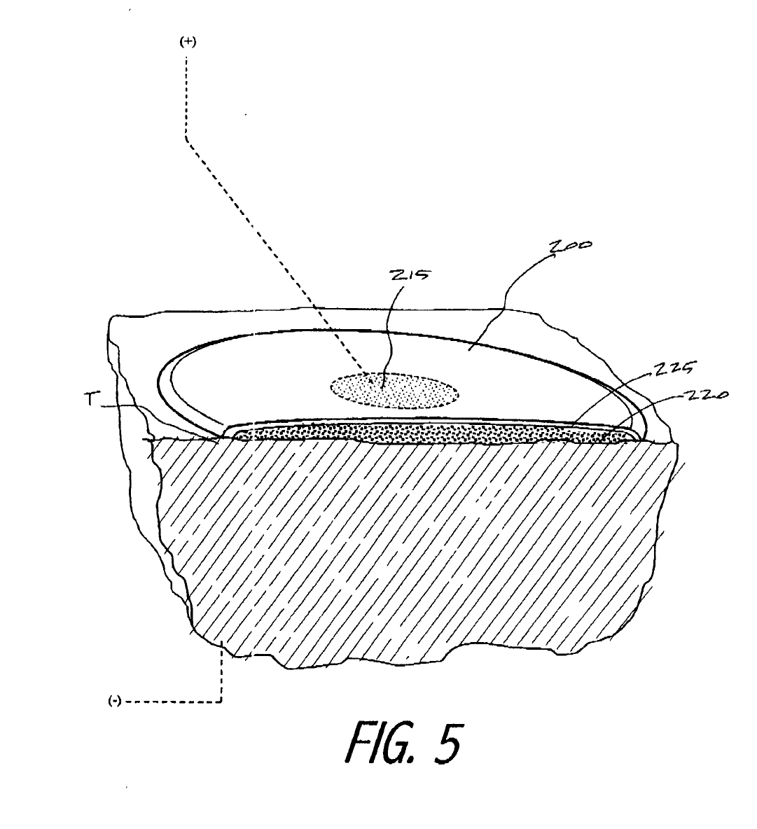

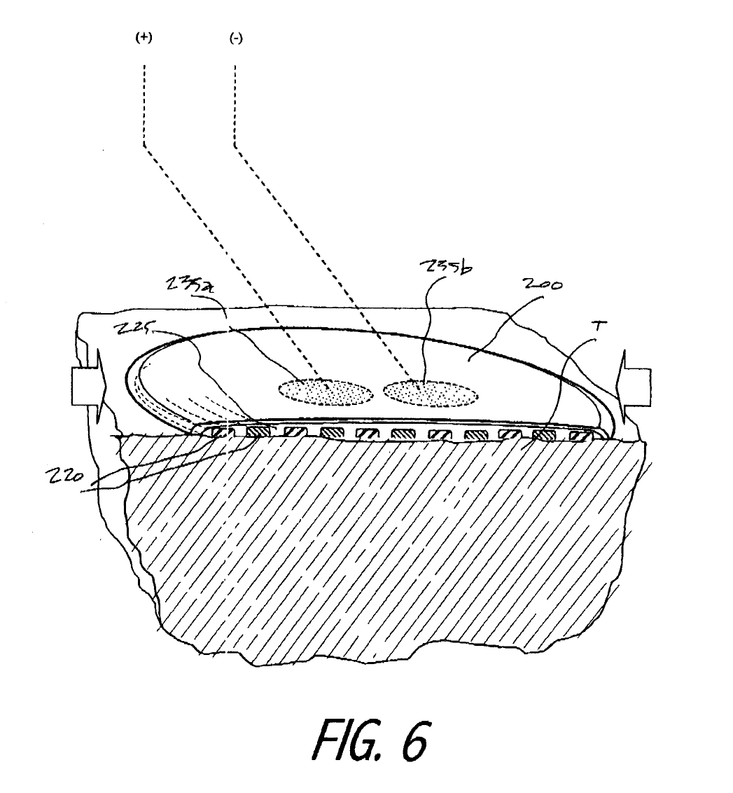

2. Type “B” embodiment of surface-conforming electrosurgical tape or pad. FIGS. 5 and 6 illustrate a Type “B” electrosurgical device in the form of patch-like surface-conforming member 200 for creating ohmic heating in a tissue surface. In this embodiment, the conforming member 200 is not directly coupled to the electrical source 140 by electrical leads as is contemplated by the Type “A” device. The conforming member 200 rather is applied and adhered to the tissue surface as an independent component. Thereafter, the distal end of an introducer with an exposed electrode contact (or contacts) is placed in substantial contact with electrode portion 215 in the otherwise insulated outer layer 225 of the patch 200 to deliver Rf energy thereto. FIG. 5 shows a first engagement layer 225 that is of a conductive flexible polymer as described previously. FIG. 6 shows a bi-polar variant of the invention. It can be seen how an introducer with bi-polar electrodes can interface with contacts 235a and 235b to apply energy to the conforming member 200.

FIG. 6 shows another aspect of the invention wherein the conforming member 200, that is, either or both the first and second surfaces 220 and 225 are at least in part a heat-shrink polymer. Thus, after the conforming member 200 is glued to the tissue surface—such as a pelvic floor—the material can be reduced in a selected dimension (see arrows in FIG. 6) by heat from the first and second surfaces 220 and 225 which effectively can be resistively heated. At the same time, the tissue can be heated for the purpose of shrinkage.

In another similar embodiment, it can be easily understood how a patch or tape having a heat-shrink capacity can be folded and adhered around a lung segment. Thereafter, the entire patch can be shrunken by heat to compress the engaged lung volume that is substantially surrounded by the tape. At the same time, the patch will seal an exterior of the lung. The compressed lung segment then will allow other lung tissue to function better—in a type of lung volume reduction surgery. The sealed, encapsulated and shrunken lung volume also can be resected to further enhance lung volume reduction.

In another embodiment, it can be understood how a patch or tape (without heat-shrink capacity) can be extended around at least a portion of a patient's vasculature to create thermal effects and lesions therein. For example, a tape device could extended about a patient's pulmonary vessels to alter conduction pathways in an endoscopic surgery, as in known in treatments for atrial fibrillation and similar disorders of electrical conduction pathways in and about the heart.

Those skilled in the art will appreciate that the exemplary embodiments and descriptions of the invention herein are merely illustrative of the invention as a whole. Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only being the true purview, spirit and scope of the invention.

What is claimed is:

1. An electrosurgical device, comprising:
a flexible conforming member;
a first engagement surface of the conforming member comprising a flexible conductive material, the engagement surface for engaging tissue; and
a second surface of the tape member comprising a flexible insulative material.

2. The electrosurgical device of claim 1 wherein the flexible conforming member is a tape or patch.

3. The electrosurgical device of claim 1 wherein the flexible conforming member is elastic.

4. The electrosurgical device of claim 1 wherein the flexible conforming member is foldable.

5. The electrosurgical device of claim 1 wherein the flexible conforming member is stretchable.

6. The electrosurgical device of claim 1 wherein the flexible conforming member is substantially of biodegradable polymers.

7. The electrosurgical device of claim 1 wherein the flexible conforming member is at least in part a heat-shrink polymer.

8. The electrosurgical device of claim 1 wherein the flexible conductive material comprises conductive filaments in a conductive plastic.

9. The electrosurgical device of claim 8 wherein the conductive plastic comprises a conductively doped polymer.

10. The electrosurgical device of claim 8 further comprising an electrical source coupled to the conductive filaments.

11. The electrosurgical device of claim 1 wherein the first surface carries a plurality of spaced apart flexible conductive portions.

12. The electrosurgical device of claim 10 wherein the plurality of spaced apart flexible conductive portions are coupled to an electrical source to define opposing polarities therein.

13. The electrosurgical device of claim 1 wherein the second surface of the conforming member is a transparent polymer.

14. The electrosurgical device of claim 1 wherein either or both the first and second surfaces of the conforming member carry a thermochromic composition.

15. The electrosurgical device of claim 1 wherein the first surface carries an adhesive composition.

16. The electrosurgical device of claim 1 wherein the first surface carries a cyanoacrylate glue.

17. The electrosurgical device of claim 1 wherein the first surface carries a fibrin-carrying glue.

18. The electrosurgical device of claim 1 wherein either or both the first and second surfaces are of a material selected from the class consisting of polysiloxanes, polyurethanes, PFTEs, polyacrylates, polyamides, polyesters, polyolefins, nylons and copolymers thereof.

19. The electrosurgical device of claim 1 further comprising a handle member for dispensing the conforming member from a distal end thereof.

20. The electrosurgical device of claim 19 further comprising a cutting member for cutting the dispensed conforming member.

21. An electrosurgical method for controlled application of energy to a surface of body structure, comprising the steps of:
(a) providing a flexible conforming member with a first engagement surface of a conductive material and a second outer surface of a flexible insulative material;
(b) adhering the conforming member to a targeted surface of the body structure, and
(c) delivering Rf energy to said first surface and thereby applying energy to said targeted surface.

22. The electrosurgical method of claim 21 wherein step (c) applies mono-polar energy to said targeted surface.

23. The electrosurgical method of claim 21 wherein step (c) applies bi-polar energy to said targeted surface.

24. The electrosurgical method of claim 21 wherein step (c) shrinks tissue to a selected depth in the targeted surface.

25. The electrosurgical method of claim 21 wherein step (c) coagulates a selected depth in the targeted tissue surface.

26. The electrosurgical method of claim 21 wherein step (c) creates a lesion to a selected depth in the targeted tissue surface.

27. The electrosurgical method of claim 21 wherein step (c) is preceded by the step of folding or wrapping the conforming member around a targeted surface of an organ.

28. The electrosurgical method of claim 21 wherein the body structure is the surface of the patient's liver, lung, spleen, pancreas, intestine, peritoneal or pre-peritoneal layer, pelvic floor or vasculature.

29. The electrosurgical method of claim 27 wherein the targeted tissue surface is the patient's pulmonary vasculature in a treatment of atrial fibrillation.

30. The electrosurgical method of claim 27 wherein the conforming member carries a pharmacological agent and a further step comprises delivery said agent to the engaged tissue surface.

31. An electrosurgical method for controlled application of energy a surface of body structure, comprising the steps of:
(a) providing a flexible conforming member with a first engagement surface of a conductive material and a second outer surface of a flexible insulative material, either or both the first and second surfaces of a heat shrink polymer;
(b) adhering the conforming member to a targeted surface of the body structure, and
(c) delivering Rf energy to the conforming member thereby shrinking a dimension of the heat shrink polymer together with a dimension of the targeted surface.

32. The electrosurgical method of claim 31 further comprising the steps of applying energy to the engaged tissue.

33. The electrosurgical method of claim 31 wherein step (c) is preceded by the step of folding or wrapping the conforming member around a surface of an organ.

34. The electrosurgical method of claim 31 wherein the organ is a lung portion and step (c) compresses the lung portion to reduce lung volume.

35. The electrosurgical method of claim 31 wherein the targeted tissue surface is the patient's pelvic floor.

36. An electrosurgical system, comprising:
a thin flexible conforming member that defines an engagement surface;
the engagement surface comprising a conductive polymer;
a tissue-adhesive carried on said engagement surface; and
an electrical source detachably coupleable to the conductive polymer.

37. The electrosurgical system of claim 36, further comprising an exterior surface of the conforming member of a flexible insulative material.
